(19) United States
(12) Patent Application Publication
(10) Pub. No.: US 2010/0049059 A1
(43) Pub. Date: Feb. 25, 2010

(54) APPARATUS AND METHOD FOR MEASURING BLOOD PRESSURE

(76) Inventors: Kiryong HA, Daejeon-city (KR); Yunkyung PARK, Daejeon-city (KR); Jeunwoo LEE, Daejeon-city (KR)

Correspondence Address: LADAS & PARRY LLP, 224 SOUTH MICHIGAN AVENUE, SUITE 1600, CHICAGO, IL, 60604, US

(21) Appl. No.: 12/510,312

(22) Filed: Jul. 28, 2009

(30) Foreign Application Priority Data
Aug. 25, 2008 (KR) 10-2008-0082817

(51) Int. Cl. A61B 5/02; A61B 5/0402

(52) U.S. Cl. 600/485; 600/509

(57) ABSTRACT

Disclosed are blood pressure measuring apparatus and method, in which a blood pressure measuring posture of a person to be examined is calculated on the basis of signals that are measured by an inclination measuring unit, so as to guide the person to be examined to maintain a reference measuring posture. When it is confirmed that the person to be examined maintains the reference measuring posture, blood pressure is measured on the basis of a measured living body signal of the person to be examined. By this disclosure, since the blood pressure measuring posture of the person to be examined can be correctly guided by inclination sensors, the person to be examined can accurately measure the blood pressure using a noninvasive and a nonpressurized method.

### RELATED APPLICATIONS

The present application claims priority to Korean Patent Application Serial Number 10-2008-0082817, filed on Aug. 25, 2008, the entirety of which is hereby incorporated by reference.

### BACKGROUND OF THE INVENTION

1. Field of the Invention

The present invention relates to an apparatus and method for measuring blood pressure, and more particularly, to an apparatus and method for measuring blood pressure that can guide a correct blood pressure measuring posture of a person to be examined.

2. Description of the Related Art

Blood pressure has been used as an important reference index in respects to determining a level of cardiovascular diseases. At the present time, as the average span of human life increases in our society and our society actually goes into an aging society, the cardiovascular diseases have increased. Accordingly, an accurate and simple blood pressure measuring technology has attracted attention.

In general, a blood pressure measuring method may be divided into an invasive method and a noninvasive method. The invasive method inserts catheters into blood vessels and directly measures blood pressure, and is mainly used to continuously monitor the blood pressure when critically ill patients are managed in places, such as operating rooms. However, the invasive method requires complicated preparations and surgical operations and high cost, and has the high possibility of developing other complications, such as tissue damaging due to contamination or closing. Thus, the invasive method is used in only some cases, such as intensive care units.

Examples of the noninvasive method include auscultatory measurement, palpatory measurement, oscillometric measurement, Doppler ultrasound measurement, volume-oscillometric measurement, and pulse wave velocity measurement. At the present time, most of the electronic blood pressure measuring apparatuses use an oscillometric method.

The oscillometric method pressurizes a cuff attached to an upper arm of a specific subject to have pressure higher than systolic blood pressure so as to block blood vessels and then searches a characteristic vibration while decreasing the pressure. However, the oscillometric method cannot continuously monitor the blood pressure and can provide an accurate blood pressure value only in the case where the circumference of a portion where the cuff is wound at the time of using the cuff is considered. In addition, the oscillometric method may damage blood vessels or tissues, because a process of applying pressure of about 200 mmHg is needed.

Most of the blood pressure measuring methods using a noninvasive blood pressure measuring apparatus based on the oscillometric method among the blood pressure measuring methods according to the related art generally use the cuff. As a result, it is not possible to continuously measure blood pressure. In order to resolve the problem that occurs when the cuff is used, a method that analyzes a waveform obtained through a photoplethysmogram or a method that obtains a pulse transit time (PTT) from an electrocardiogram and a PPG signal and calculates blood pressure using the pulse transit time has been suggested.

The blood pressure measuring method using the pulse transit time (PTT) is a method that is noninvasive and can measure blood pressure without pressurizing through the cuff. The blood pressure measuring method using the pulse transit time (PTT) is the best technology in terms of continuous blood pressure measurement, miniaturization of a blood pressure measuring apparatus, and convenient blood pressure measurement. This blood pressure measuring method anticipates a numerical value of blood pressure on the basis of a correlation between the pulse transit time and the blood pressure, on the assumption that blood is transmitted from the heart to capillary vessels of fingers or the like through a hard tube. In actuality, since blood vessel walls (arteries) have small elasticity of 0.0018 liter/mmHg on average, the above assumption is sufficiently possible.

As such, various attempts to accurately and easily measure blood pressure have been suggested though various methods. At the present time, most important elements that are required to widely use a portable blood pressure measuring apparatus include convenient measurement, miniaturization of a measuring apparatus, and measurement of continuous blood pressure values. However, the above methods have a problem in that measurement results are incorrect. Shortage of accuracy results from not only an error due to deviation of individual persons in the blood pressure measuring method using the pulse transit time but also an error due to inaccuracy of the posture of wearing the measuring apparatus caused by miniaturization of the measuring apparatus.

### SUMMARY OF THE INVENTION

Accordingly, it is an object of the present invention to provide an apparatus and method for measuring blood pressure that can calculate the posture of a person to be examined using inclination sensors and guide the person to be examined to maintain an accurate blood pressure measuring posture, thereby accurately measuring blood pressure using a pulse transit time (PTT).

According to an embodiment of the present invention, there is provided a blood pressure measuring apparatus that measures blood pressure of a person to be examined. The blood pressure measuring apparatus includes: an inclination measuring unit that includes inclination sensors, and measures an inclination of the blood pressure measuring apparatus from signals that are detected by the inclination sensors; a living body signal measuring unit that receives a living body signal from the person to be examined and measures an electrocardiogram and a pulse wave; and a central processing unit that calculates a blood pressure measuring posture of the person to be examined on the basis of the signals measured by the inclination measuring unit, determines whether the person to be examined maintains a predetermined reference measuring posture by comparing the calculated blood pressure measuring posture of the person to be examined and the predetermined reference measuring posture, and measures the blood pressure of the person to be examined on the basis of the living body signal that is measured by the living body signal measuring unit. The inclination sensors are acceleration sensors or three-axis gravity acceleration sensors.

The inclination measuring unit measures an elevation angle that is an angle between the surface of the earth and the blood pressure measuring apparatus and a rotation angle of the blood pressure measuring apparatus, on the basis of the signals that are detected by the inclination sensors. At this time, the central processing unit calculates a height difference between the heart of the person to be examined and the blood pressure measuring apparatus on the basis of the elevation angle and height information of the person to be examined, and calculates the blood pressure measuring posture of the person to be examined on the basis of the calculated height difference between the heart of the person to be examined and the blood pressure measuring apparatus. The central processing unit sets a reference rotation angle on the rotation angle, compares the reference rotation angle and the rotation angle measured by the inclination measuring unit, and calculates the blood pressure measuring posture of the person to be examined according to whether the measured rotation angle corresponds to the reference rotation angle.

The central processing unit outputs data that is used to guide the person to be examined to maintain the reference measuring posture, when it is determined that the person to be examined does not maintain the reference measuring posture on the basis of the elevation angle and the rotation angle that are measured by the inclination measuring unit. The central processing unit outputs a driving signal to the living body signal measuring unit, when it is determined that the person to be examined maintains the reference measuring posture on the basis of the elevation angle and the rotation angle that are measured by the inclination measuring unit.

The blood pressure measuring apparatus further includes a user interface unit that receives at least one of body information including age, sex, height, and weight of the person to be examined, and a display unit that outputs a blood pressure measurement result of the central processing unit, a blood pressure measuring posture calculation result of the person to be examined, and data used to guide the person to be examined to maintain the reference measuring posture.

According to another embodiment of the present invention, there is provided a blood pressure measuring method that measures blood pressure of a person to be examined using a blood pressure measuring apparatus. The blood pressure measuring method includes receiving signals detected by inclination sensors that are provided in the blood pressure measuring apparatus; calculating a blood pressure measuring posture of the person to be examined on the basis of the signals input through the inclination sensors and comparing the calculated blood pressure measuring posture of the person to be examined and a predetermined reference measuring posture; and detecting a living body signal of the person to be examined in accordance with the comparison result and measuring the blood pressure of the person to be examined on the basis of the detected living body signal. In this case, the inclination sensors are acceleration sensors or three-axis gravity acceleration sensors.

The comparing of the calculated blood pressure measuring posture of the person to be examined and the predetermined reference measuring posture includes measuring an elevation angle that is an angle between the surface of the earth and the blood pressure measuring apparatus and a rotation angle of the blood pressure measuring apparatus, on the basis of the signals that are detected by the inclination sensors. The blood pressure measuring method further includes calculating a height difference between the heart of the person to be examined and the blood pressure measuring apparatus on the basis of the elevation angle and height information of the person to be examined, and the blood pressure measuring posture of the person to be examined is calculated on the basis of the calculated height difference between the heart of the person to be examined and the blood pressure measuring apparatus. The blood pressure measuring method further includes confirming whether the rotation angle corresponds to a reference rotation angle, and the blood pressure measuring posture of the person to be examined is calculated according to whether the rotation angle corresponds to the reference rotation angle.

The comparing of the calculated blood pressure measuring posture of the person to be examined and the predetermined reference measuring posture includes outputting a blood pressure measuring posture calculation result of the person to be examined and data that is used to guide the person to be examined to maintain the reference measuring posture.

The measuring of the blood pressure includes measuring an electrocardiogram and a pulse wave signal on the basis of the living body signal of the person to be examined to calculate a pulse transit time, and the blood pressure of the person to be examined is measured on the basis of the pulse transit time.

According to the present invention, a blood pressure measuring posture of a person to be examined can be calculated using inclination sensors so as to guide the person to be examined to maintain a correct blood pressure measuring posture. Accordingly, since the person to be examined can measure his or her blood pressure at the correct blood pressure measuring posture, accurate blood pressure measurement is enabled.

Further, since a blood pressure measuring apparatus is implemented in a band type to have portability, the person to be examined can easily carry the blood pressure measuring apparatus and measure blood pressure, without depending on a time and a place.

Further, since an external transmission module is provided, the person to be examined can actively recognize his or her health state by transmitting his or her blood pressure measurement result to the outside.

### BRIEF DESCRIPTION OF THE DRAWINGS

FIG. 1 is a block diagram illustrating the configuration of a blood pressure measuring apparatus according to the present invention;

FIGS. 2A and 2B are perspective views illustrating a blood pressure measuring apparatus according to an embodiment of the present invention;

FIGS. 3A to 6B are diagrams illustrating the operation of a blood pressure measuring apparatus according to an embodiment of the present invention; and

FIG. 7 is a flowchart illustrating an operational flow of a blood pressure measuring method according to another embodiment of the present invention.

### DESCRIPTION OF THE PREFERRED EMBODIMENTS

Hereinafter, the preferred embodiments of the present invention will be described with reference to the accompanying drawings.

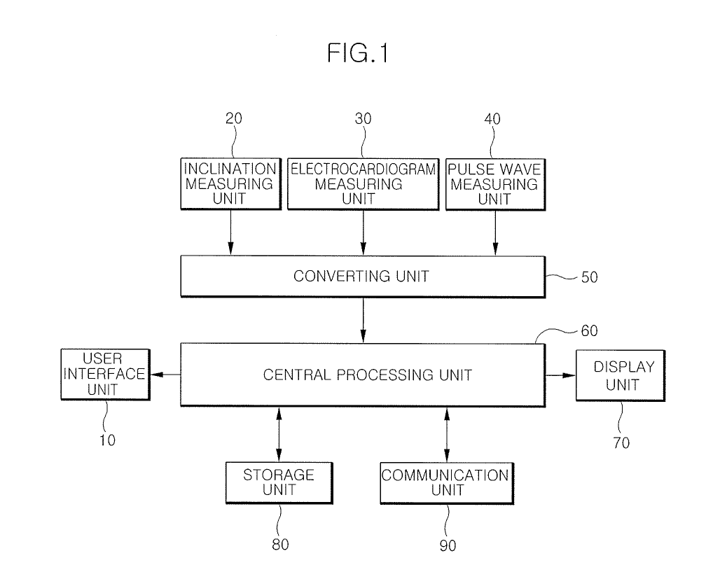

FIG. 1 is a block diagram illustrating the configuration of a blood pressure measuring apparatus according to the present invention.

FIG. 1 is a diagram that is referred to in order to describe the configuration of an information security managing apparatus of a wireless terminal. Referring to FIG. 1, a blood pressure measuring apparatus according to the present invention includes a user interface unit 10, an inclination measuring unit 20, a living body signal measuring unit, a converting unit 50, a central processing unit 60, a display unit 70, a storage unit 80, and a communication unit 90.

The user interface unit 10 receives body information of a person to be examined, such as age, sex, height, and weight, and can be configured using a key button or a touch screen. The display unit 70 includes an LCD or a touch screen, and displays measured data, such as an electrocardiogram, a pulse wave, and blood pressure, which is measured by the central processing unit 60. In this case, in the case where the display unit 70 is the touch screen, the user interface unit 10 and the display unit 70 may be integrally formed.

The storage unit 80 stores the body information of the person to be examined that is input from the user interface unit 10, and the measured data, such as an electrocardiogram, a pulse wave, and blood pressure, which is measured by the central processing unit 60. The communication unit 90 includes at least one of a wireless communication module, such as Bluetooth, ZigBee, and a wireless LAN, and a wired communication module, such as a serial interface and a USB, and exchanges data with an external apparatus using a wired/wireless communication method. At this time, the communication unit 90 transmits to the outside signals that are measured by the inclination measuring unit 20 and the living body signal measuring unit and the measured data, such as an electrocardiogram, a pulse wave, and blood pressure measured by the central processing unit 60.

The inclination measuring unit 20 measures a motion of the person to be examined, when blood pressure is measured using the blood pressure measuring apparatus. At this time, the inclination measuring unit 20 includes an inclination sensor. In this case, the inclination sensor is an acceleration sensor, and the acceleration sensor can measure two axes or more of acceleration signals and measure the acceleration of gravity.

Preferably, the inclination measuring unit 20 includes a three-axis gravity acceleration sensor. In this case, the three-axis gravity acceleration sensor measures the acceleration of gravity in three-axis directions, and measures acceleration values in x-axis, y-axis, and z-axis directions, respectively. For example, when a simple motion is made to vertically erect the blood pressure measuring apparatus or rotate the blood pressure measuring apparatus upward and downward, the three-axis gravity acceleration sensor can measure the acceleration in the three-axis directions, thereby outputting an azimuth angle changed due to an inclination.

Accordingly, the inclination measuring unit 20 uses the three-axis gravity acceleration sensor to measure an elevation angle that is an angle between a direction where an arm of the person to be examined extends and the surface of the earth, and a rotation angle that indicates a degree to which a wrist rotates on the basis of the direction where the arm extends. At this time, in order to remove a minute motion value due to hand trembling or noise, a signal that is detected by the inclination sensor passes through a low-pass filter. Then, the elevation angle and the rotation angle are calculated.

The central processing unit 60 can calculate a blood pressure measuring posture of the person to be examined, on the basis of the data that is measured by the inclination measuring unit 20. At this time, the central processing unit 60 determines whether the calculated blood pressure measuring posture of the person to be examined is maintained at a reference measuring posture. When it is determined that the person to be examined does not maintain the reference measuring posture, the central processing unit 60 outputs a voice or a message that urges the person to be examined to take the reference measuring posture, thereby guiding a correct blood pressure measuring posture. Preferably, the central processing unit 60 guides the blood pressure measuring posture of the person to be examined, such that a blood pressure measured portion of the person to be examined is horizontal to the surface of the earth, the height of the blood pressure measured portion is equal to the height of the heart, and fingers come into contact with the blood pressure measuring apparatus in a vertical direction. Meanwhile, the central processing unit 60 activates the operation of the living body signal measuring unit, when the calculated blood pressure measuring posture of the person to be examined is maintained at the reference measuring posture.

The living body signal measuring unit receives a living body signal of the person to be examined and measures a pulse wave and an electrocardiogram, and includes an electrocardiogram measuring unit 30 and a pulse wave measuring unit 40. In this case, the pulse wave is assumed as a photoplethysmography (hereinafter, referred to as ‘PPG’).

In this case, the electrocardiogram measuring unit 30 includes two or more sensors to measure an electrocardiogram (ECG), and detects an electrocardiogram signal on the basis of signals that are detected by the two or more sensors. Meanwhile, the pulse wave measuring unit 40 detects a PPG signal of the person to be examined. Since the PPG includes a heart rate, blood oxygen saturation, and contraction and expansion of blood vessels, a blood vessel state of the person to be examined can be measured through the PPG.

The converting unit 50 converts the electrocardiogram signal and the PPG signal, which are measured by the living body signal measuring unit, from an analog signal to a digital signal. The central processing unit 60 measures a pulse transit time (PTT), on the basis of the electrocardiogram signal and the PPG signal that are converted by the converting unit 50 from the analog signals to the digital signals. The central processing unit 60 uses the measured pulse transit time and the body information of the person to be examined, such as age, sex, height, and weight, which is input from the user interface unit 10, to calculate a pulse wave velocity (PWV), thereby measuring blood pressure of the person to be examined.

At this time, the central processing unit 60 measures systolic blood pressure and diastolic blood pressure, and the measured blood pressure is output to a screen through the display unit 70 or transmitted to an external apparatus through the communication unit 90.

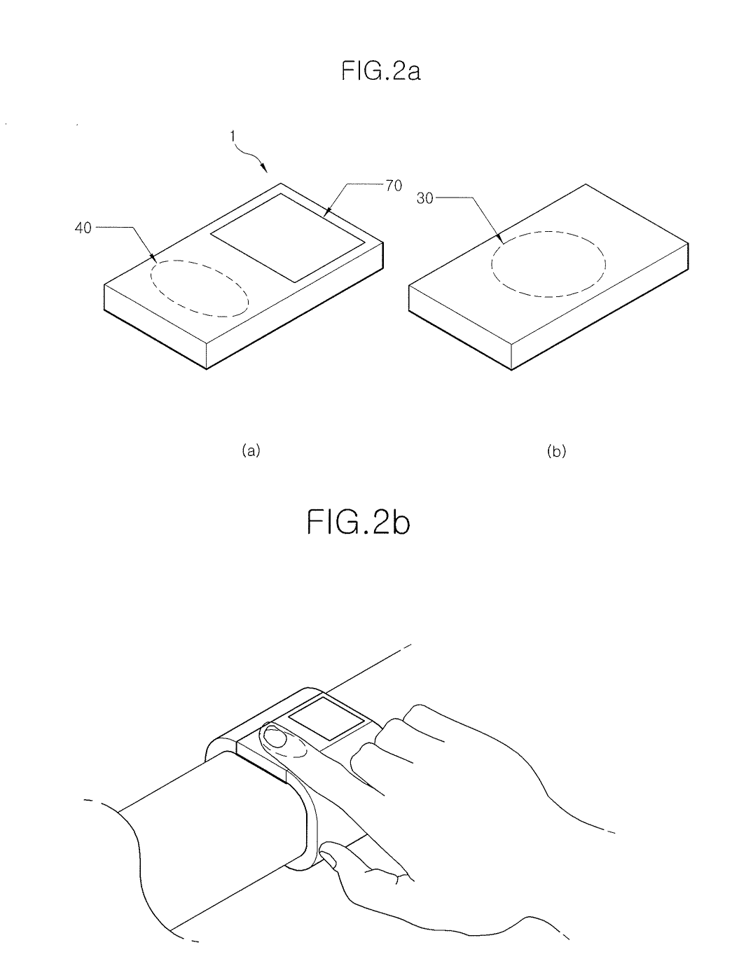

FIGS. 2A and 2B are diagrams illustrating a blood pressure measuring apparatus according to an embodiment of the present invention. Specifically, FIGS. 2A and 2B are perspective views illustrating a main body of a blood pressure measuring apparatus.

As shown in FIG. 2A, the blood pressure measuring apparatus according to the embodiment of the present invention includes a display unit 70 that is provided at an upper portion of the main body 1 and outputs measured data, and sensors of the electrocardiogram measuring unit 30 and the pulse wave measuring unit 40. At this time, the sensors of the electrocardiogram measuring unit 30 and the pulse wave measuring unit 40 may be disposed in one area, such that the electrocardiogram signal and the PPG signal can be measured, when one finger comes into contact with the sensors. However, the present invention is not limited thereto, and the sensors of the electrocardiogram measuring unit 30 and the pulse wave measuring unit 40 may be disposed in different areas, respectively, such that the electrocardiogram signal and the PPG signal can be measured through different fingers, respectively. Further, another sensor of the electrocardiogram measuring unit 30 is provided at a lower portion of the main body 1.

In this case, the blood pressure measuring apparatus according to the embodiment of the present invention is implemented in a band type so as to be worn on a wrist of the person to be examined, as shown in FIG. 2B. At this time, the electrocardiogram measuring unit 30 that is provided at the lower portion of the main body 1 comes into contact with a skin of the wrist of the person to be examined who wears the blood pressure measuring apparatus, and the electrocardiogram measuring unit 30 and the pulse wave measuring unit 40 that are provided at the upper portion of the main body 1 open upward, enabling other fingers of the person to be examined to come into contact with the sensors of the electrocardiogram measuring unit 30 and the pulse wave measuring unit 40.

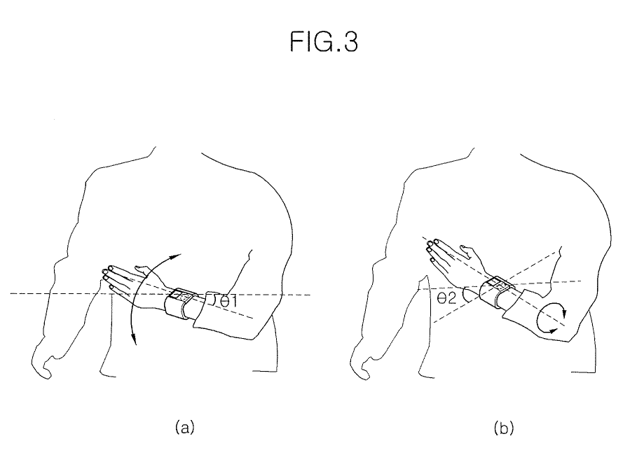

FIGS. 3A to 5 are diagrams illustrating the operation of a blood pressure measuring apparatus according to an embodiment of the present invention.

FIGS. 3A and 3B show the operation of measuring an elevation angle and a rotation angle according to a motion of a person to be examined, in a state where the person to be examined wears a blood pressure measuring apparatus.

FIG. 3A shows the operation of measuring an elevation angle. In this case, an elevation angle that the blood pressure measuring apparatus (direction where an arm extends) forms on the basis of the surface of the earth when the person to be examined upward and downward moves the arm where the blood pressure measuring apparatus is worn, that is, ‘θ1’ is measured. FIG. 3B shows the operation of measuring a rotation angle. In this case, a rotation angle of the blood pressure measuring apparatus when the person to be examined rotates the wrist leftward and rightward while using an extension direction of the arm where the blood pressure measuring apparatus is worn as a rotation axis, that is, ‘θ2’ is measured.

By using the above-described methods, if the elevation angle ‘θ1’ and the rotation angle ‘θ2’ of the blood pressure measuring apparatus are measured, the central processing unit 60 can use the elevation angle and the rotation angle to calculate a height difference between the blood pressure measuring apparatus and the heart of the person to be examined. Of course, in order to accurately calculate a relative height difference with the heart, sensors may need to be additionally provided around the heart. In this invention, it is assumed that the blood pressure is measured at the posture where the person to be examined sits down, without providing additional hardware, for convenient portability and easy measurement.

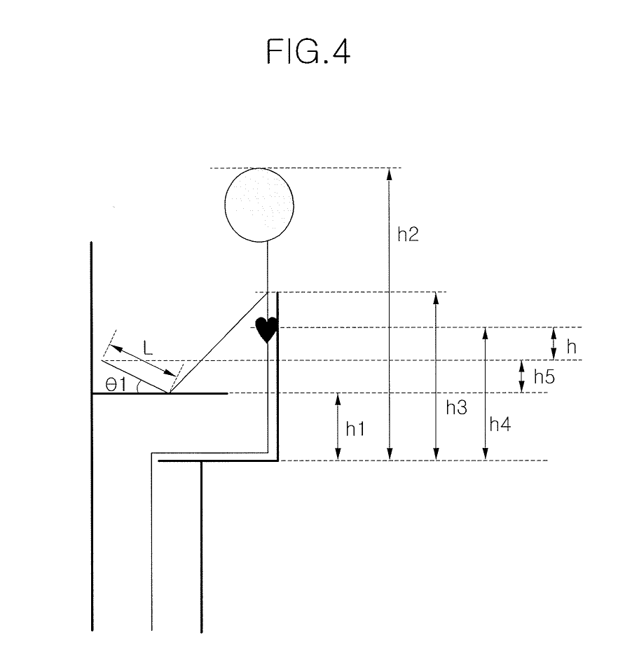

The height difference between the blood pressure measuring apparatus according to the embodiment of the present invention and the heart of the person to be examined can be calculated with reference to the embodiment shown in FIG. 4.

FIG. 4 shows a process of calculating a height difference between the blood pressure measuring apparatus according to the embodiment of the present invention and the heart.

The blood pressure measuring apparatus according to the embodiment of the present invention can use the fact when the height and the arm length has a correlation therebetween, the height and the sitting height has a correlation therebetween, and the height and the shoulder height has a correlation therebetween to store average body dimensional data, and calculate the height difference between the blood pressure measuring apparatus and the heart using an elevation angle, on the basis of the stored average body dimensional data. That is, the height difference between the blood pressure measuring apparatus and the heart can be calculated on the basis of the height of the person to be examined and the elevation angle.

At this time, it is assumed that the blood pressure of the person to be examined is measured in a state where the person to be examined sits down, in order to accurately measure data. In addition, it is assumed that the blood pressure measuring apparatus is driven in a state where the person to be examined sits at a chair and lays an elbow on a desk. In this case, the shoulder height, the sitting height, and the arm length can be calculated from the height of the person to be examined, on the basis of the existing standard human body sizes.

Referring to FIG. 4, ‘h1’ denotes a height difference between the chair and the desk, and the height difference is provided as a predetermined constant value, but may be changed according to setting. In this embodiment, it is assumed that ‘h1=25 cm’ is set.

In addition, ‘h2’ denotes the sitting height of the person to be examined. If height information is input from the person to be examined, the sitting height can be calculated on the basis of the input height information. That is, on the assumption that the height and the sitting height are at 1:0.54 on the basis of the existing standard human body sizes, if the height is 175 cm, the sitting height can be calculated as 94.5 cm.

In addition, ‘h3’ denotes the height of the person to be examined from the hip to the shoulder in a state where the person to be examined sits at the chair. If the height information is input from the person to be examined, the corresponding height can be calculated on the basis of the input height information. That is, on the assumption that the height and the shoulder height are at 1:0.8 on the basis of the existing standard human body sizes, if the height is 175 cm, the shoulder height can be calculated as 140 cm.

Accordingly, ‘h3’ can be calculated (h3=59.5 cm) by subtracting a sum between the shoulder height (140 cm) and the sitting height (94.5 cm) by the height (175 cm).

In addition, ‘h4’ denotes the height of the person to be examined from the hip to the heart in a state where the person to be examined sits at the chair, and can be calculated from ‘h3’. That is, on the assumption that ‘h3:h4=1:0.75’ is satisfied on the basis of the existing standard human body sizes, since h3 is 59.5 cm, h4=0.75×59.5 cm≈44.6 cm can be calculated.

In addition, ‘h5’ denotes the height from the desk on which the elbow of the person to be examined lays to the blood pressure measuring apparatus that the person to be examined wears on the wrist, which is measured using the elevation angle θ1. The height ‘h5’ can be calculated using the elevation angle θ1 and the height information input from the person to be examined. At this time, the length of the lower portion of the arm (length L from the wrist to the elbow) can be calculated from the height information that is input from the person to be examined. That is, on the assumption that the height and the arm length are at 1:0.34 on the basis of the existing standard human body sizes, if the height is 175 cm, the arm length can be calculated as 59.5 cm. At this time, if it is assumed that the arm length is 59.5 cm and the arm length and the length of the lower portion of the arm are at 1:0.43, the length of the lower portion of the arm can be calculated as L≈25.6 cm.

Meanwhile, an equation ‘h5=L×sin θ1’ can be derived using an equation ‘sin θ1=h5/L’. At this time, a value of ‘h5’ changes according to the elevation angle ‘θ1’. For example, in the case of ‘θ1=22°’, h5=25.6×sin 22°≈9.6 cm is obtained.

Accordingly, the height difference between the heart of the person to be examined and the blood pressure measuring apparatus can be calculated from an equation ‘h=h4−(h1+h5). At this time, h=44.6−(25+9.6)=10 cm is obtained.

In this case, the central processing unit 60 generates a message of ‘please raise your wrist above 10 cm’ in consideration of the height difference between the heart and the blood pressure measuring apparatus that is 10 cm, and allows the generated message to be output through the display unit 70. Of course, the central processing unit 60 may generate voice data and output the generated voice data through a speaker (not shown).

By the above method, if the height difference between the heart of the person to be examined and the blood pressure measuring apparatus is in a range of ‘h=0’ or ‘−α<h<α’, the central processing unit 60 determines that the blood pressure measuring posture using the elevation angle θ1 is the reference measuring posture.

Meanwhile, the central processing unit 60 sets a reference rotation angle β for the rotation angle θ2. When the rotation angle θ2 measured by the inclination measuring unit 20 corresponds to the reference rotation angle β, the central processing unit 60 determines that the blood pressure measuring posture using the rotation angle θ2 is the reference measuring posture.

Of course, when the rotation angle θ2 measured by the inclination measuring unit 20 does not correspond to the reference rotation angle β, the central processing unit 60 generates a message of ‘please rotate your wrist inward by 10° and allows the generated message to be output through the display unit 70.

In this case, the reference rotation angle β is preferably based on an angle that is vertical to the surface of the earth. However, the present invention is not limited thereto, and the reference rotation angle may be changed according to setting.

At this time, the blood pressure measuring apparatus according to the embodiment of the present invention measures a PPG signal of a finger location. In this case, since the PPG signal is vulnerable to pressure, it is important to determine whether the finger is accurately located vertical to the blood pressure measuring apparatus. For this purpose, in this invention, the rotation angle is used to determine whether the blood pressure measuring apparatus and the finger of the person to be examined are accurately located in a vertical direction.

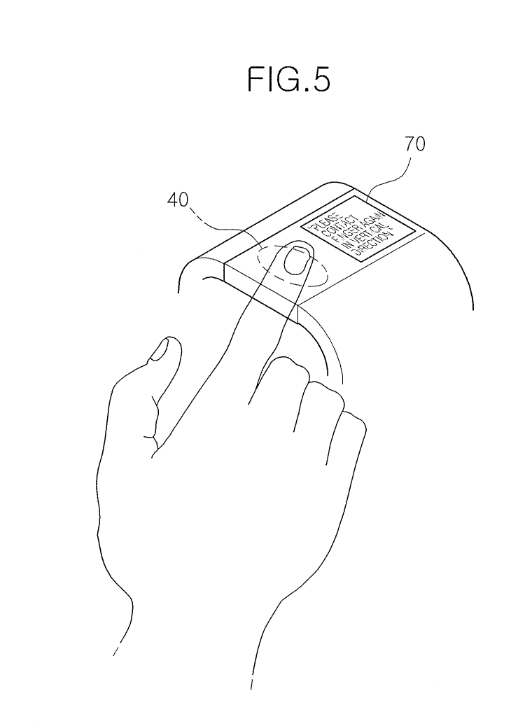

FIG. 5 shows an example of when a message is displayed on a screen of a display unit 70, when a person to be examined does not maintain a reference measuring posture. That is, when it is determined that the height of the blood pressure measuring apparatus or a direction where the blood pressure measuring apparatus and the finger of the person to be examined form is not matched with the reference rotation angle β, the central processing unit 60 outputs a message informing that the height or direction is not matched with the reference rotation angle through the screen of the display unit 70, as shown in FIG. 5, thereby guiding the person to be examined to maintain the reference measuring posture.

Accordingly, when it is determined that the height of the blood pressure measuring apparatus that the person to be examined wears is equal to the height of the heart and the finger and the blood pressure measuring apparatus are correctly disposed to correspond to the reference rotation angle β, the central processing unit 60 outputs control commands to the electrocardiogram measuring unit 30 and the pulse wave measuring unit 40, such that the electrocardiogram signal and the PPG signal of the person to be examined are detected and the operation of measuring blood pressure is performed.

At this time, the central processing unit 60 outputs control commands to allow the electrocardiogram measuring unit 30 and the pulse wave measuring unit 40 to be driven, only when it is determined that both the blood pressure measuring posture using the elevation angle θ1 and the blood pressure measuring posture using the rotation angle θ2 are correct.

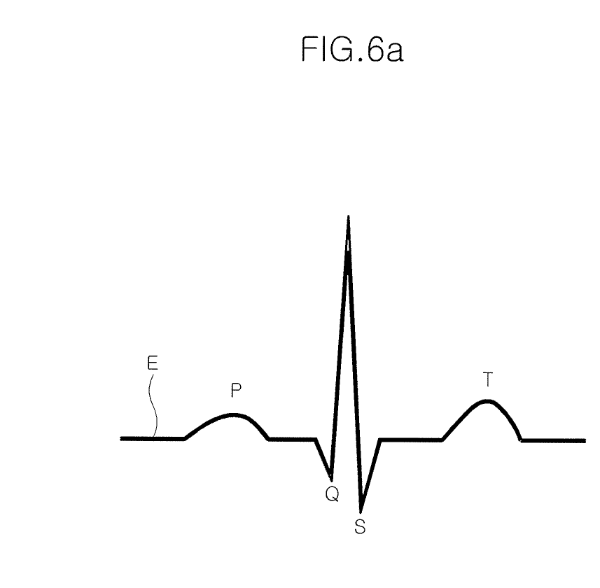

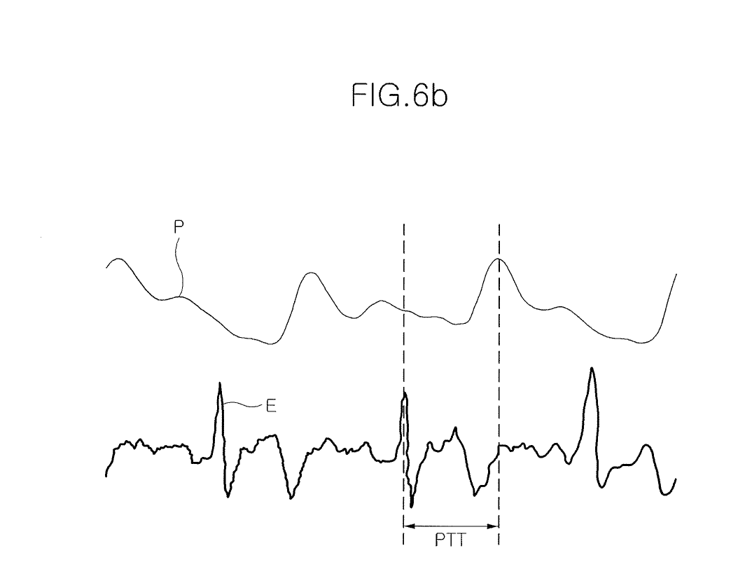

FIGS. 6A and 6B show waveforms of an electrocardiogram signal and a PPG signal.

FIG. 6A shows a waveform of an electrocardiogram signal. In this case, a waveform of an electrocardiogram signal E shows a record of electrical signals for periodical contraction and expansion of the heart. This waveform shows a record of main electrical signals that are generated in the heart, and is divided into P, Q, R, S, and T waves in accordance with locations of individual peaks. Among them, R that is a maximum peak point is intimately associated with a point of time when blood starts to be emitted from the heart.

Accordingly, in this embodiment, R as the maximum peak point is used as a point of time when the blood starts to be emitted from the heart. That is, as shown in FIG. 6B, the pulse transit time PTT is measured by simultaneously measuring the electrocardiogram signal E and the PPG signal P and setting the maximum peak point R of the electrocardiogram signal E as a start point and the maximum value point of the PPG signal P as an arrival point.

In this case, the pulse transit time PTT is time that is needed until the blood starts from the heart and arrives at the fingertip. A point of time when the blood starts to be emitted from the heart can be recognized from the electrocardiogram signal, and a point of time when the blood arrives at the fingertip can be recognized by observing a change in oxygen saturation of the blood flowing through the fingertip through the PPG signal.

Meanwhile, information of the person to be examined, such as age, sex, height, and weight, is used as variables that are used to calculate the blood pressure together with the pulse transit time. First, the height of the person to be examined is used when the correct measuring posture is guided by calculating the height difference between the blood pressure measuring location and the heart, and the information of the person to be examined, such as age, sex, and weight, is used when determining a constant value to improve accuracy of a blood pressure value.

The operation of the present invention that has the above-described configuration is as follows.

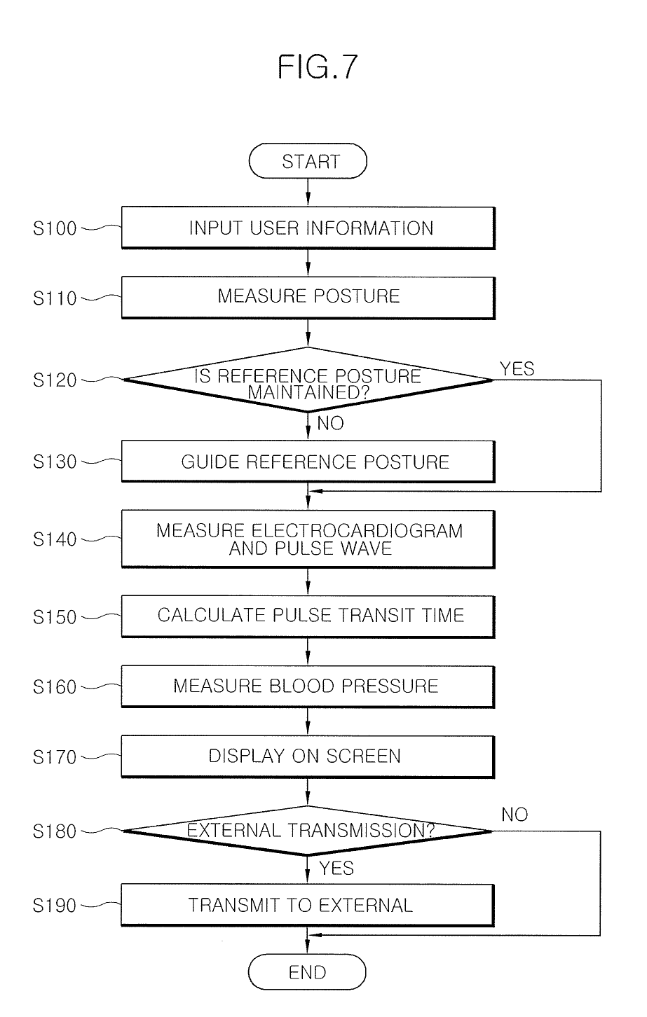

FIG. 7 shows an operational flow of a blood pressure measuring method according to another embodiment of the present invention.

Referring to FIG. 7, if the user information is input from the user interface unit 10 (S100), the acceleration sensor of the inclination measuring unit 20 operates. Accordingly, a motion of the person to be examined is detected and an elevation angle and a rotation angle are measured, and the posture of the person to be examined is measured through the measured elevation angle and rotation angle (S110). At this time, the central processing unit 60 determines whether the person to be examined maintains the reference measuring posture. When it is determined that the person to be examined does not maintain the reference measuring posture (S120), the central processing unit 60 outputs a voice or a message, thereby guiding the person to be examined to maintain the reference measuring posture (S130). The related embodiment has been described with reference to FIGS. 3 to 5.

Meanwhile, when it is determined that the person to be examined maintains the reference measuring posture (S120), the central processing unit operates the sensors of the electrocardiogram measuring unit 30 and the pulse wave measuring unit 40 to measure an electrocardiogram and a pulse wave of the person to be examined (S140), thereby allowing the pulse transit time PPT to be calculated through the electrocardiogram and the pulse wave (S150). Accordingly, the central processing unit 60 measures the blood pressure of the person to be examined using the calculated pulse transit time (S160), and displays a measured value on the screen of the display unit 70 (S170).

When it is required to output the measured value to the outside (S180), the central processing unit 60 transmits the measured value to an external apparatus connected through the communication unit 90 (S190).

As described above, the apparatus and method for measuring blood pressure according to the embodiments of the present invention have been described with reference to the exemplary drawings. However, the present invention is not limited to the embodiments and the drawings disclosed in this specification, and various changes and medications can be made without departing from the spirit and scope of the present invention.

What is claimed is:

1. A blood pressure measuring apparatus that measures blood pressure of a person to be examined, comprising:
an inclination measuring unit that includes inclination sensors, and measures an inclination of the blood pressure measuring apparatus from signals that are detected by the inclination sensors;
a living body signal measuring unit that receives a living body signal from the person to be examined and measures an electrocardiogram and a pulse wave; and
a central processing unit that calculates a blood pressure measuring posture of the person to be examined on the basis of the signals measured by the inclination measuring unit, determines whether the person to be examined maintains a predetermined reference measuring posture by comparing the calculated blood pressure measuring posture of the person to be examined and the predetermined reference measuring posture, and measures the blood pressure of the person to be examined on the basis of the living body signal that is measured by the living body signal measuring unit.

2. The blood pressure measuring apparatus of claim 1,
wherein the inclination sensors are acceleration sensors.

3. The blood pressure measuring apparatus of claim 1,
wherein the inclination sensors are three-axis gravity acceleration sensors.

4. The blood pressure measuring apparatus of claim 1,
wherein the inclination measuring unit measures an elevation angle that is an angle between the surface of the earth and the blood pressure measuring apparatus and a rotation angle of the blood pressure measuring apparatus, on the basis of the signals that are detected by the inclination sensors.

5. The blood pressure measuring apparatus of claim 4,
wherein the central processing unit calculates a height difference between the heart of the person to be examined and the blood pressure measuring apparatus on the basis of the elevation angle and height information of the person to be examined, and calculates the blood pressure measuring posture of the person to be examined on the basis of the calculated height difference between the heart of the person to be examined and the blood pressure measuring apparatus.

6. The blood pressure measuring apparatus of claim 4,
wherein the central processing unit sets a reference rotation angle on the rotation angle, compares the reference rotation angle and the measured rotation angle, and calculates the blood pressure measuring posture of the person to be examined according to whether the measured rotation angle corresponds to the reference rotation angle.

7. The blood pressure measuring apparatus of claim 4,
wherein the central processing unit outputs data that is used to guide the person to be examined to maintain the reference measuring posture, when it is determined that the person to be examined does not maintain the reference measuring posture on the basis of the elevation angle and the rotation angle that are measured by the inclination measuring unit.

8. The blood pressure measuring apparatus of claim 4,
wherein the central processing unit outputs a driving signal to the living body signal measuring unit, when it is determined that the person to be examined maintains the reference measuring posture on the basis of the elevation angle and the rotation angle that are measured by the inclination measuring unit.

9. The blood pressure measuring apparatus of claim 1,
wherein the living body signal measuring unit includes:
an electrocardiogram measuring unit that includes two or more sensors and detects an electrocardiogram signal; and
a pulse wave measuring unit that detects a photoplethysmography signal.

10. The blood pressure measuring apparatus of claim 1, further comprising:
a user interface unit that receives at least one of body information including age, sex, height, and weight of the person to be examined.

11. The blood pressure measuring apparatus of claim 1, further comprising:
a display unit that outputs a blood pressure measurement result of the central processing unit, a blood pressure measuring posture calculation result of the person to be examined, and data used to guide the person to be examined to maintain the reference measuring posture.

12. A blood pressure measuring method that measures blood pressure of a person to be examined using a blood pressure measuring apparatus, comprising:
receiving signals detected by inclination sensors that are provided in the blood pressure measuring apparatus;
calculating a blood pressure measuring posture of the person to be examined on the basis of the signals input through the inclination sensors and comparing the calculated blood pressure measuring posture of the person to be examined and a predetermined reference measuring posture; and
detecting a living body signal of the person to be examined in accordance with the comparison result and measuring the blood pressure of the person to be examined on the basis of the detected living body signal.

13. The blood pressure measuring method of claim 12,
wherein the inclination sensors are acceleration sensors.

14. The blood pressure measuring method of claim 12,
wherein the inclination sensors are three-axis gravity acceleration sensors.

15. The blood pressure measuring method of claim 12,
wherein the comparing of the calculated blood pressure measuring posture of the person to be examined and the predetermined reference measuring posture includes:
measuring an elevation angle that is an angle between the surface of the earth and the blood pressure measuring apparatus and a rotation angle of the blood pressure measuring apparatus, on the basis of the signals that are detected by the inclination sensors.

16. The blood pressure measuring method of claim 15, further comprising:
calculating a height difference between the heart of the person to be examined and the blood pressure measuring apparatus on the basis of the elevation angle and height information of the person to be examined,
wherein the blood pressure measuring posture of the person to be examined is calculated on the basis of the calculated height difference between the heart of the person to be examined and the blood pressure measuring apparatus.

17. The blood pressure measuring method of claim 15, further comprising:
confirming whether the rotation angle corresponds to a reference rotation angle,
wherein the blood pressure measuring posture of the person to be examined is calculated according to whether the rotation angle corresponds to the reference rotation angle.

18. The blood pressure measuring method of claim 12,
wherein the comparing of the calculated blood pressure measuring posture of the person to be examined and the predetermined reference measuring posture includes:
outputting a blood pressure measuring posture calculation result of the person to be examined and data that is used to guide the person to be examined to maintain the reference measuring posture.

19. The blood pressure measuring method of claim 12, further comprising:
before receiving the signals,
receiving at least one of body information including age, sex, height, and weight of the person to be examined.

20. The blood pressure measuring method of claim 12,
wherein the measuring of the blood pressure includes:
measuring an electrocardiogram and a pulse wave signal on the basis of the living body signal of the person to be examined to calculate a pulse transit time,
the blood pressure of the person to be examined is measured on the basis of the pulse transit time.
